(19) United States
(12) Patent Application Publication
(10) Pub. No.: US 2024/0424200 A1
(43) Pub. Date: Dec. 26, 2024

(54) VEIN GRAFT PREPARATION PUMP

(71) Applicant: Quest Medical, Inc., Allen, TX (US)

(72) Inventors: Steve Parnis, Missouri City, TX (US); Andrew Nelson, Dallas, TX (US); Jeffrey Albertsen, McKinney, TX (US)

(21) Appl. No.: 18/749,002

(22) Filed: Jun. 20, 2024

Related U.S. Application Data
(60) Provisional application No. 63/522,552, filed on Jun. 22, 2023.

(51) Int. Cl. A61M 5/145

(52) U.S. Cl. CPC A61M 5/1454; A61M 5/14546; A61M 2210/125

(57) ABSTRACT

The disclosed principles provide a vein graft preparation pump, and related methods of use and manufacturing thereof, providing a constant and uniform pressure of fluid flowed through a vein graft being prepared for use in a bypass procedure. An exemplary pump includes a syringe holder having an opening for holding a syringe body, and a slot formed within the opening for receiving a flange of the syringe body. The syringe holder also includes left and right side rails, and a spring housing coupled to rear ends of the side rails to secure the rear end of a spring therein. A plunger depressor includes a back in contact with the front end of the spring, and a front having a plunger base for engaging a syringe plunger. Decompression of the spring moves the plunger depressor along the syringe holder during use of the pump thereby providing constant and uniform pressure depressing said syringe plunger.

### RELATED APPLICATIONS

This disclosure claims priority to U.S. Provisional Patent Application Ser. No. 63/522,552, filed Jun. 22, 2023, the entirety of which is incorporated herein by reference for all purposes.

### TECHNICAL FIELD

This disclosure relates generally to the field of vein grafting, and more particularly to a novel vein graft preparation pump for preparing veins for grafting, as well as methods of use and methods of manufacture thereof.

### BACKGROUND

Coronary artery bypass surgery, also called coronary artery bypass grafting (CABG) is a surgical procedure to treat coronary artery disease (CAD), which is, generally speaking, the buildup of plaque in the arteries of the heart. CABG and percutaneous coronary intervention (PCI) are the two methods to revascularize stenotic lesions of the cardiac arteries. PCI is a non-surgical procedure used to treat narrowing of the coronary arteries using a combination of coronary angioplasty with stenting. Conversely, a CABG procedure is a surgical procedure performed to bypass narrowing areas of the heart's arteries by using arteries or veins harvested from other parts of the body. After performing such a bypass, CABG restores adequate blood supply to the heart, thereby slowing the progression of CAD and increasing life expectancy for the patient.

A common CABG procedure involves a saphenous vein graft (SVG) for the bypassing of narrowed areas of the arteries. The great saphenous vein (or long saphenous vein) is a large, subcutaneous, superficial vein of the leg. It is the longest vein in the body, running along the length of the lower limb for returning blood from the foot, leg and thigh to the deep femoral vein. However, long-term patency (i.e., remaining sufficiently open) of SVGs has reported vein graft failure (VGF) is as high as 45% at 18 months after surgery. It is believed that such high, long-term failure rate is because preparation of SVGs before grafting leads to injury of the vein prior to grafting, which may promote VGF.

Some structural changes of the tunica intima (i.e., the innermost layer) to prevent thickening (i.e., intimal hyperplasia) and vein wall remodeling are necessary for vein graft adaptation to the arterial environment. During and after the harvesting, veins go through a period of ischemia (restriction in blood flow/supply) and reperfusion (tissue damage caused when blood supply returns to tissue after a period of lack of oxygen) after engraftment, which causes damage to endothelial cells (cells that line the interior surface of the vein) and smooth muscle cells (the cells present in the layer surrounding the tunica intima).

Veins are naturally adapted to an environment of low pressure and low blood flow; however, in a graft, veins are transferred and integrated into the arterial circulation, where they are exposed to high pressure and flow. This exposure to arterial pressure and flow that causes increased shear stress and wall tension, which further damages the endothelial layer and smooth muscle cells. Over time, such continued damage results in luminal loss that makes the graft more susceptible to atherosclerosis. Progressive atherosclerosis is the primary cause of late vein graft failure.

Conventional preparation of saphenous veins for an SVG typically involves the manual distension (i.e., stretching) of the veins using fluid pressure. For example, U.S. Pat. No. 3,958,557 provides a device and method for preparing a blood vessel, such as the saphenous vein, for use as a coronary bypass graft. FIG. 1 illustrates an isometric view of a cannula 10 constructed in accordance with the principles disclosed in the U.S. Pat. No. 3,958,557. This conventional cannula 10 is comprised of a medical grade polyethylene having the flexibility and softness required to prevent trauma to the intima of the vein in which it is used. The cannula 10 has a hub 14 at the proximal end thereof which is cylindrical and terminates in a planar surface 16. The hub 14 is connected to a body 12 that is diametrically enlarged to form a shoulder 18 in a plane parallel to the surface 16. The hub 14 is sized to accommodate coupling with surgical tubing (not shown), which is press-fit on the hub 14 and abuts shoulder 18. The body 12 is frustoconical in configuration, the forwardly tapering ramp surface 20 merging with stylet 22, which forms the distal end of the cannula 10. The stylet 22 has a very gradual taper from the body 12 to the distal end 24. The external dimension of the stylet 22 is selected to be essentially the normal internal diameter of a suitable vein segment for use in coronary bypass. The stylet 22 includes a peripheral flange 26 spaced slightly behind the distal end 24. The peripheral flange 26 has a forwardly tapering ramp surface 28 that facilitates insertion of the stylet 22 into a suitable vein segment 30. The flange 26 terminates in a shoulder 32 which permits pressure-tight ligation, as described below.

FIG. 2 illustrates a side, partial cross-section view of the conventional cannula 10 taught in U.S. Pat. No. 3,958,557. The stylet 22 has an annular bore 34 that opens at the distal end 24 and is also in open communication with hollow 38 in the hub 14. The hollow 38 is coaxial with the bore 34 and is provided with a Luer taper so as to form a female coupling. Thus, the cannula 10 is press-coupled to an irrigation instrument such as a syringe 40. The selected portion of the vein 30 is then severed and the stylet 22 inserted within the lumen of the resected vein 30 until the vein 30 is pressed tightly upon the ramp surface 20. Thereafter, temporary ligation is effected by tying suture 42 tightly around the vein 30 near the flange 26. Ligation will have the effect of creating a fluid seal between the vein segment 30 and the cannula 10.

Thereafter, a syringe 40 is used for irrigating purposes to determine whether occlusions or clots remain in the vein segment 30 and, if so, to accommodate flushing of the vein 30. A distant portion of the vein segment 30 can be clamped and manual hydrostatic pressure communicated to the vein through the cannula 10 with the syringe 40. This applied pressure is used to detect leaks in the vein segment 30, particularly at the severed tributary sites. This pressure also serves to determine the distensibility index of the vein 30. If the vein 30 distends too easily under pressure, it is not suitable for the coronary artery bypass. Assuming the vein 30 has been proven under this manual pressurization, the vein 30 is used for the coronary artery bypass.

However, data has shown that acute damage to the vein graft intima can result from extensive distension of such veins during repair of the severed tributaries of the vein graft, but that this does not necessarily occur at pressure equivalent to normal arterial pressure. However, manual pressure distension does not limit or otherwise regulate the pressure of fluid applied and does not circulate the fluid during preparation. Consequently, intimal damage, although unknown to the clinician at the time, is often caused to the grafted vein when prepared using such manual procedures due to high pressurization as a result of unregulated manual pressurization techniques found in U.S. Pat. No. 3,958,557 as well as other conventional approaches. Accordingly, what is needed in the art is a device and related method for limiting the pressure of the vein graft during preparation for a vein graft to be used in a coronary artery bypass procedure, that does not suffer from the deficiencies found with conventional vein preparation approaches. The disclosed principles provide such a unique device, methods of use, and methods of manufacture thereof.

### SUMMARY

The disclosed principles prevent over pressurization of veins harvested for vein grafts by controlling the amount and the consistency of pressure applied through the harvested veins from a continuous flow of fluid through the vein graft. This combination of regulated pressurization and continuity/uniformity in the pressure applied through harvested veins is provided using a unique vein graft preparation pump constructed in accordance with the disclosed principles. Generally speaking, the apparatus incorporates a spring with a set spring constant (K) that is sufficient to deliver fluids from a syringe through a vessel cannula inserted into the vein section selected for the grafting procedure. Additionally, the disclosed apparatus provides only a limited amount of pressure such that no injury is caused to the vein. Pressure and flow is limited and maintained constant via use of the spring, i.e., no pressure changes/spikes, which provides uniform pressurization at a predetermined amount safe for the harvested vein section, thereby eliminating the human error present with manual pressurization during distension of grafted veins.

In one embodiment, a vein graft preparation pump in accordance with the disclosed principles may comprise a syringe holder having a front end and a back end, the front end having a forward-facing opening configured to hold a syringe body and a slot formed within the forward-facing opening configured to receive a flange at a base of the syringe body. The syringe holder may also include a body comprising substantially parallel left side and right side rails connected to the front end and extending rearward from the front end, and a support proximate the back end connecting rear ends of left side and right side rails. Such an exemplary pump may also comprise a spring having a front end and a rear end, and a spring housing coupled to the rear ends of the left side and right side rails and configured to secure the rear end of the spring therein. The pump may also include a plunger depressor having a back in contact with the front end of the spring, and a front having a plunger base configured to engage a distal end of a syringe plunger, wherein the plunger depressor is configured to laterally move from the front end to the back end of the syringe holder. Moreover, when the plunger depressor is moved proximate the rear end of the syringe holder, the pump is in a loaded state where the spring is compressed between the plunger depressor and the spring housing, and during use of the pump, decompression of the compressed spring causes movement of the plunger depressor towards the front end of the syringe holder thereby providing constant and uniform pressure depressing the syringe plunger.

Additional embodiments and advantages and variation thereof are also encompassed within the scope of the disclosed principles, and some such exemplary embodiments discussed in further detail herein.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawing, in which:

FIG. 1 illustrates an isometric view of a cannula constructed in accordance with the prior art, and used in the irrigation of a vein harvested for a grafting procedure;

FIG. 2 illustrates a side, partial cross-section view of the conventional cannula illustrated in FIG. 1;

FIG. 3 illustrates an isometric view of one embodiment of a vein graft preparation pump designed and constructed in accordance with the disclosed principles;

FIG. 4 illustrates an isometric view of one embodiment of a syringe holder for use with the vein graft preparation pump illustrated in FIG. 1, designed and constructed in accordance with the disclosed principles;

FIG. 5 illustrates an isometric view of one embodiment of a spring housing for use with the vein graft preparation pump illustrated in FIG. 1, designed and constructed in accordance with the disclosed principles;

FIG. 6 illustrates an isometric view of one embodiment of a spring for use with the vein graft preparation pump illustrated in FIG. 1, designed and constructed in accordance with the disclosed principles;

FIG. 7 illustrates an isometric view of one embodiment of a plunger depressor for use with the vein graft preparation pump illustrated in FIG. 1, designed and constructed in accordance with the disclosed principles;

FIG. 8 illustrates an isometric view of one embodiment of a lock handle for use with the vein graft preparation pump illustrated in FIG. 1, designed and constructed in accordance with the disclosed principles;

FIG. 9 illustrates a side view of the vein graft preparation pump illustrated in FIG. 1 in a loaded, unused state; and

FIG. 10 illustrates a side view of the vein graft preparation pump illustrated in FIG. 1 in a deployed, used state.

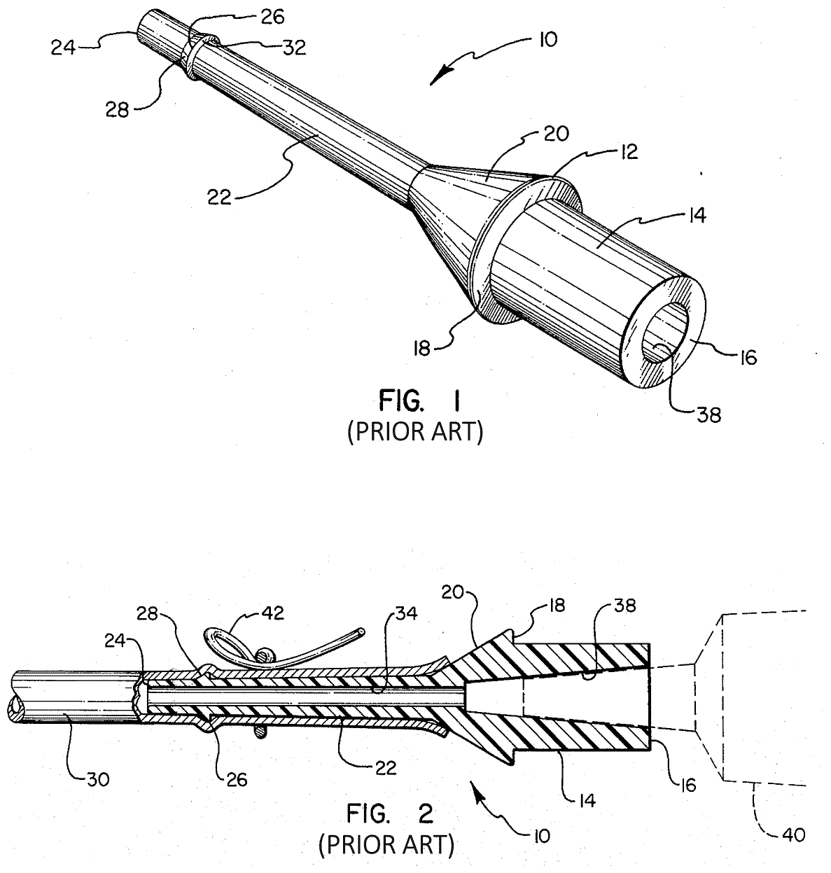

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Although multiple embodiments are shown and discussed in great detail, it will be apparent to those skilled in the relevant art that some features that are not relevant to the present invention may not be shown for the sake of clarity.

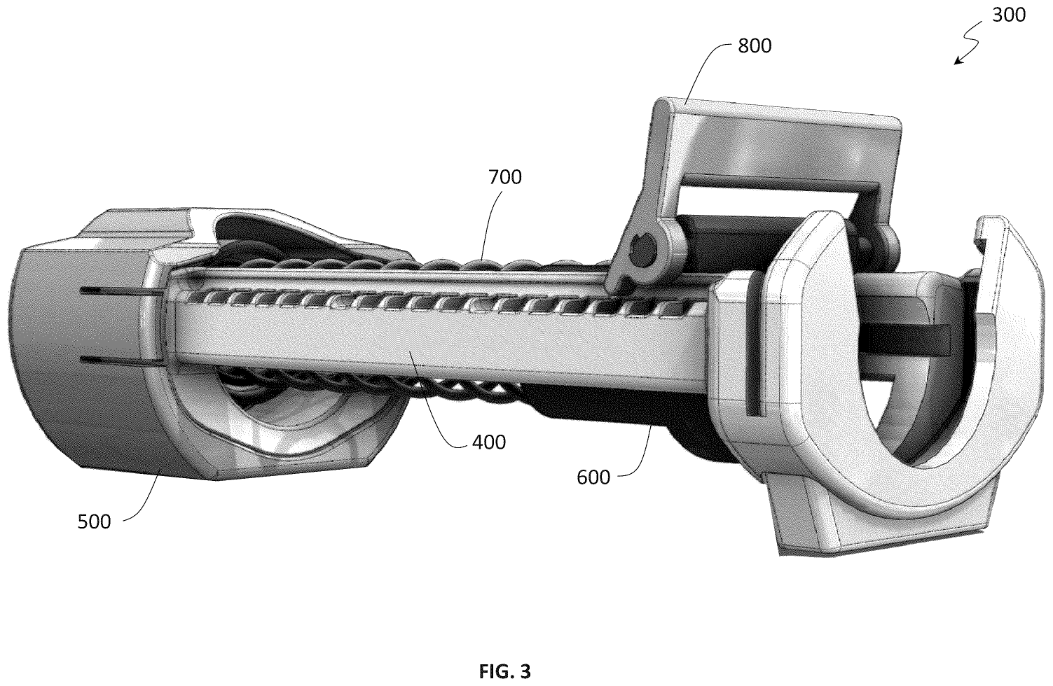

Looking now at FIG. 3, illustrated is an isometric view of one embodiment of a vein graft preparation pump 300 designed and constructed in accordance with the disclosed principles. As mentioned above, the vein graft preparation pump 300 provides a combination of regulated pressurization and consistency in the pressure applied via a fluid pushed through harvested veins. This uniform pressurization at a predetermined amount safe for the harvested vein section eliminates the human error that too often caused over pressurization due to manual pressurization during distension of grafted veins.

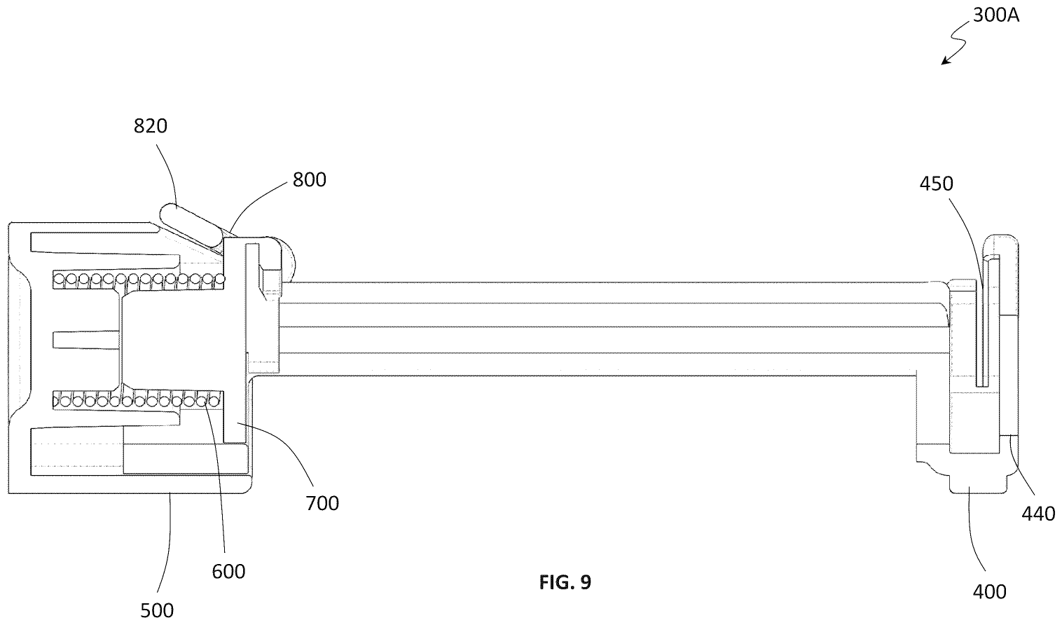

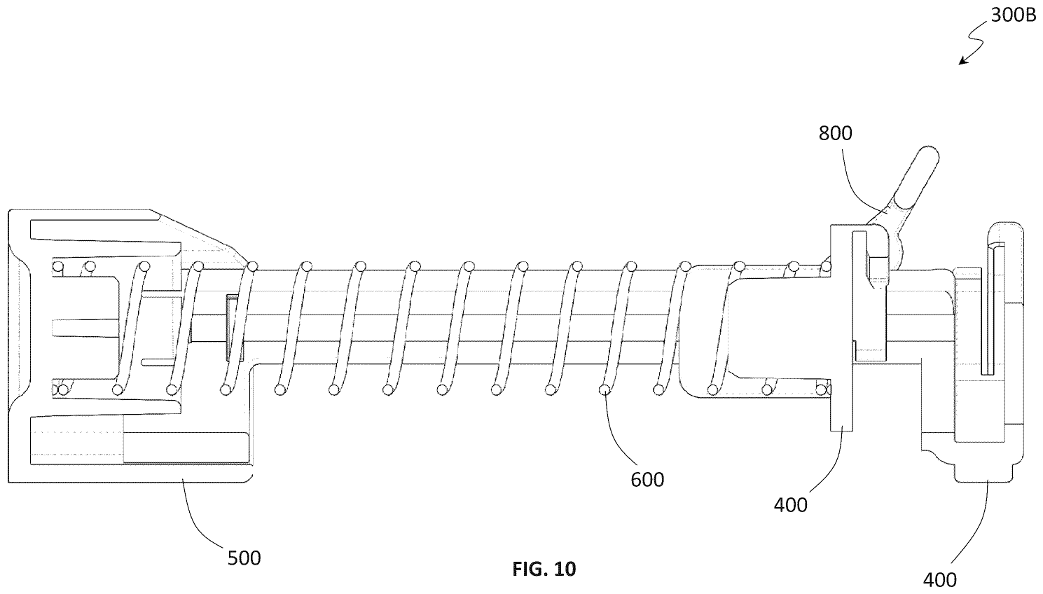

The components of the illustrated embodiment of the vein graft preparation pump 300 initially include a syringe holder 400. The syringe holder 400 is sized and configured to securely hold a syringe used to irrigate a harvested vein section with fluid during the distension process. The vein graft preparation pump 300 also includes a spring housing 500. The spring housing 500 is configured to securely retain a spring 600 in place, and which is used to provide the consistent fluid pressure by the vein graft preparation pump 300. The vein graft preparation pump 300 also includes a plunger depressor 700 configured to engage the distal end of a syringe plunger in order to depress the plunger during use of the pump 300. Finally, this embodiment of the vein graft preparation pump 300 includes a lock 800. As described in greater detail below, the lock 800 is used to permit the pump 300 to depress the plunger of a syringe during use of the pump 300, as well as to stop the depression of the plunger by the pump 300 at any time by a user. The lock 800 is also used to initially “load” the pump 300 for use, also as described below. Each of these components are discussed in greater detail below. Additionally, the functionality of a vein graft preparation pump 300 as disclosed herein is described in detail with reference to FIGS. 9 and 10 below.

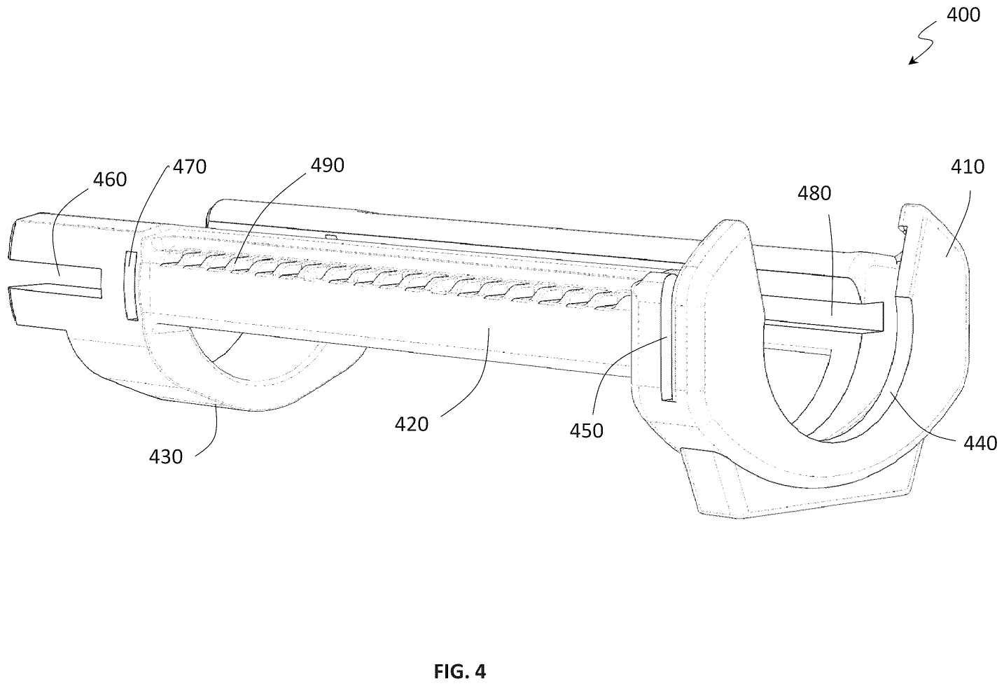

Turning now to FIG. 4, illustrated is an isometric view of one embodiment of a syringe holder 400 for use with the vein graft preparation pump 300 illustrated in FIG. 3, designed and constructed in accordance with the disclosed principles. The illustrated embodiment of the syringe holder 400 includes a front end 410 of the holder, as well as left side and right side rails 420 extending rearward from the front end 410 and forming the body of the vein graft preparation pump 300. The syringe holder 400 also incudes a support 430 at the rear end of the syringe holder 400, which provides a structural connection between the rails 420 at the back end of the syringe holder 400 to keep the rails 420 level and parallel to one another.

Within the front end 410 of the syringe holder 400 is a forward-facing opening 440, which is sized to receive the body of a syringe (not illustrated) to be used to irrigate a vein harvested for a grafting procedure. Additionally, the front end 410 includes a slot 450 sized to receive the flange found at the base of typical syringe bodies. Such a syringe flange is received within the slot 450, while the body of the syringe is held within the opening 440, so that the syringe is steadily held in place while being used with the vein grafting preparation pump 300. The syringe holder 400 further includes alignment slots 460 at its rear end, and corresponding to the back of each of the rails 420. The alignment slots 460 are used to secure and align the vein grafting preparation pump 300 within the spring housing 500, which is described in further detail below. Locking slots 470 are also included at the rear end of the syringe holder 400 for locking the spring housing 500 onto the syringe holder 400, as is also described further below.

The syringe holder 400 further includes left side and right side slide slots 480, each formed on an interior of the corresponding left side and right side rails 420. These slide slots 480, which extend substantially coextensively with the side rails 420, are sized and configured to receive corresponding guides (not illustrated) formed on the plunger depressor 700, which are described in detail below. These slide slots 480 are formed parallel to one another and are aligned at the same height within the syringe holder 400. Also, the side rails 420 each include locking features 490 formed along the tops of each of the side rails 490, and extending substantially along the length of the side rails 420. These locking features 490 are sized and configured to work with the lock 800, which is coupled to the plunger depressor 700, as described in detail below.

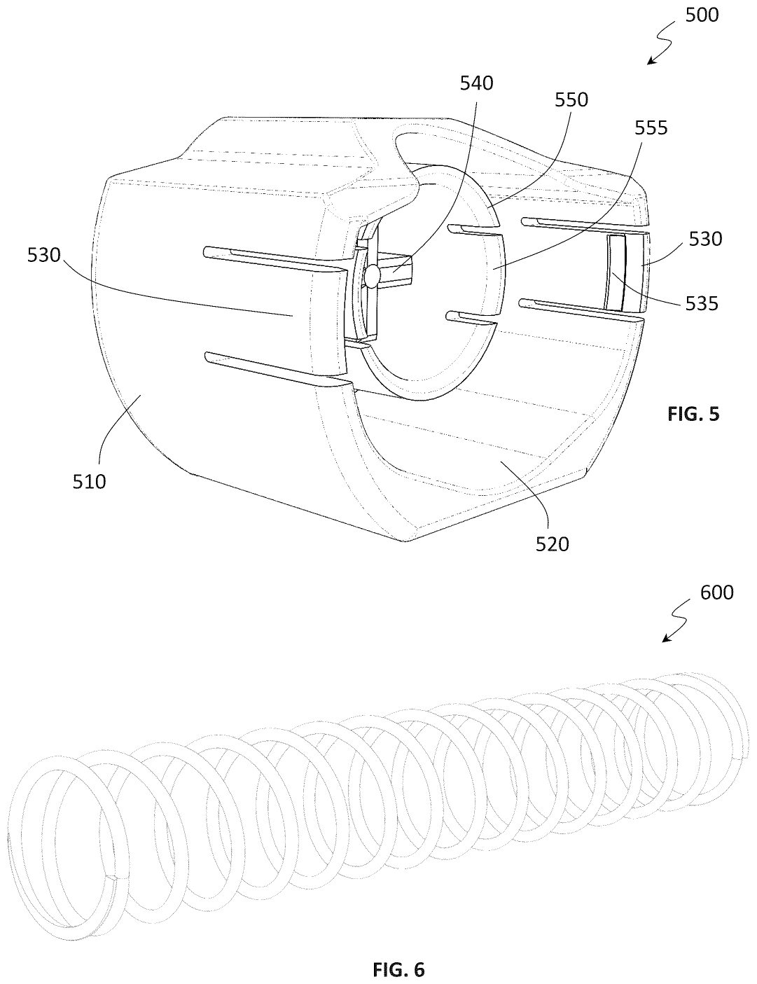

Referring now to FIG. 5, illustrated is an isometric view of one embodiment of a spring housing 500 for use with the vein graft preparation pump 100 illustrated in FIG. 1, designed and constructed in accordance with the disclosed principles. The spring housing 500 includes a body 510 having an interior cavity 520 formed within the body 510. The interior cavity 520 is sized and shaped to receive the rear end and the support 430 of the syringe holder 400 therein. As the rear end of the syringe holder 400 is being received into the spring housing 500, opposing locking arms 530 are configured to flex outwardly to permit passage of the rear end into the interior cavity 520. As the rear end is fully seated within the spring housing 500, locking tabs 535 formed on opposing, interior ends of the locking arms 530 are located so as to be received within corresponding locking slots 470 formed on the syringe holder 400 so as to securely lock the spring housing 500 onto the rear end of the syringe holder 400.

The spring housing 500 also includes lateral supports 540 extending through a spring seat 550 and extending outwardly to the exterior walls of the spring housing 500. Portions of the lateral supports 550 are sized to be received within the alignment slots 460 in order to assist in aligning the syringe holder 400 and preventing it from rotating during use of the vein grafting preparation pump 300. The spring seat 550 is sized to receive the inner diameter of the spring 600 around it. Additionally, spring locks 555 are formed at the front of the spring seat 550. These spring locks 555 are configured to securely engage one or more coils of the spring 600 to hold the spring 600 within the spring housing 500. FIG. 6 illustrates an isometric view of one embodiment of the spring 600 for use with the vein graft preparation pump 100. As mentioned above, the interior diameter of the spring 600 is sized to be received around a spring seat 550 formed within the interior cavity 520 of the spring housing 500. The length and tension of the spring 600 is selected to provide a specific amount of continuous pressure during use of the vein grafting preparation pump 300, as is described in further detail below.

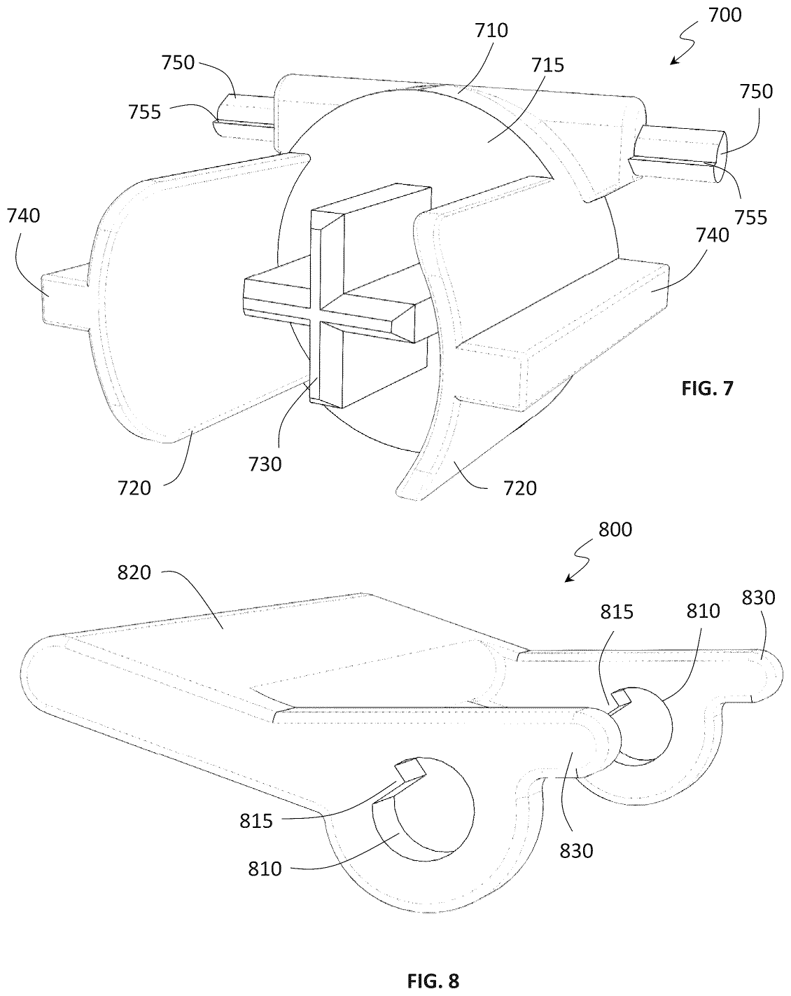

Turning now to FIG. 7, illustrated is an isometric view of one embodiment of a plunger depressor 700 for use with the vein graft preparation pump 100 illustrated in FIG. 1, designed and constructed in accordance with the disclosed principles. The plunger depressor 700 is comprised of a main body 710, which includes a plunger base 715 sized and configured to be orthogonal to the distal end of the plunger of a syringe (not illustrated) such that the distal end of the plunger may rest on the plunger base 715.

The plunger depressor 700 further includes a pair of opposing sidewalls 720 designed to secure the distal end of a syringe plunger to the plunger depressor 700 in concert with a raised feature 730 sized and shaped to be received within such a distal end of a plunger. Specifically, many distal ends of conventional syringe plungers typically include a uniquely shaped recess formed inwardly on the distal end. In most cases, this recess is an “+” shape, typically corresponding to the cross-sectional shape of the body of the syringe plunger. Providing the raised feature 730 with substantially the same shape and size as the recess formed in conventional syringe plungers results in the raised feature 730 being press-fit into such a recess. Thus, the syringe plunger may be held securely onto the plunger depressor 700. Additionally, the opposing sidewalls 720 are advantageously curved with the space between them sized to surround the outer diameter of a typical syringe plunger so as to further prevent lateral movement of the syringe plunger during was of the preparation pump 300.

The plunger depressor 700 also includes guiderails 740 extending outwardly from opposing sides of the plunger depressor 700. In this embodiment, the plunger depressor 700 includes two guiderails 740 extending at approximately 180 degrees (i.e., horizontally opposing), and these guiderails 740 are sized and located to be received in the alignment slot 460 of each of the side rails 420 of the syringe holder 400 introduced above. The thickness of each guiderail 740 and corresponding alignment slots 460 are sized for a slip-fit connection of the two, such that the plunger depressor 700 slides smoothly back-and-forth along the rails 420 of the syringe holder 400 within the alignment slots 460. This sliding movement is used to push an affixed syringe plunger into its syringe during use of the preparation pump 300, as described further below.

Also included on the plunger depressor 700 are pivot axles 750 for carrying the lock 800. This embodiment of the plunger depressor 700 includes two pivot axles 750, which are horizontally opposed and outwardly extend from a top area of the body 710. Also in this illustrated embodiment, each of the pivot axles 750 include a corresponding axle stops 755 formed on the exterior surface of each pivot axle 750. These axle stops 755 provide a positive or firm stop for the rotation of the lock 800 for the preparation pump 300. Although in this embodiment the axle stops 755 are formed as raised steps extending along a length of each pivot axle 750, any advantageous form or shape of the axle stops 755 may also be employed with a preparation pump designed and constructed in accordance with the disclosed principles.

Looking now at FIG. 8, illustrated is an isometric view of one embodiment of a lock 800 for use with the vein graft preparation pump 100 illustrated in FIG. 1, designed and constructed in accordance with the disclosed principles. The lock 800 includes pivot holes 810 formed on corresponding arms extending from a handle portion 820 graspable by a user of the preparation pump 300. The pivot holes 810, in this illustrated embodiment, are aligned along the same axis of rotation, and correspond to the pair of pivot axles 750 formed on the plunger depressor 700 discussed above. As such, the lock 800 pivots or partially rotates about the pivot axles 750 and along this axis of rotation during its use.

The lock 800 further includes pivot stops 815 formed on an interior surface of each of the pivot holes 810. These pivot stops 815 are configured to contact the corresponding axle stops 755 formed on the pivot axles 750 of the plunger depressor 700. Specifically, as the lock 800 rotates, via the pivot holes 810 being mounted on the corresponding pivot axles 750, pivot stops 815 in each of the pivot holes 810 are aligned to contact a corresponding axle stop 755 to prevent rotation of the lock 800 in direction being rotated. Moreover, the positioning of the pivot stops 815 and axle stops 755 are selected such that when the pivot stops 815 engage the axle stops 755, the rotation of the lock 800 is stopped in a position such that a locking tab 830 extending from each of the arms of the lock 800 is engaged within one of the locking features 490 formed on the rails 420 of the preparation pump 300. As such, the size and shape of the locking tabs 830 are selected to as to match the size and shape of the locking features 490, and where engagement of the two locks the sliding movement of the plunger depressor 700 in a specific location with respect to the rails 420 of the syringe holder 400. A more detailed explanation of the functionality of the exemplary preparation pump 300 is provided with reference to FIGS. 9 and 10 below.

FIG. 9 illustrates a side view of the vein graft preparation pump 100 illustrated in FIG. 1 in a loaded, unused state. In this side view, the syringe holder 400 of the pump 300 is shown on the right side of the drawing, while the spring housing 500, spring 600, plunger depressor 700, and lock 800 are shown assembled on the left side of the drawing.

In the illustrated loaded, unused state, the plunger depressor 700 is shown slid all the way to the rearmost end of the syringe holder 400 along the rails 420. In this position, the spring 600 is fully compressed in the space between the spring housing 500 and the plunger depressor 700. Additionally, the lock 800 is positioned in the locked position, where the locking tab 830 extending from each of the arms of the lock 800 is engaged within a corresponding one of the locking features 490 formed on the rails 420 of the preparation pump 300. More specifically, to load the preparation pump 300, the user may manually slide the plunger depressor 700 from the front of the pump 300 to the rearmost position, thereby compressing the spring 600. To do so, the lock 800 is placed in the unlocked position by, in this embodiment, rotating it rearward. In the unlocked position, the locking tabs 830 are removed from any engagement within the locking features 490, thus permitting sliding movement of the plunger depressor 700. In some uses, the plunger depressor 700 may be slid back to a location that is less than the fully rear location, if desired. Once the plunger depressor 700 is slid to the desired location along the rails 420, the user may then use their thumb or another digit to contact the handle portion 820 to operate the lock 800 by pivoting it from an unlocked position to the locked position. In the locked position of the lock 800, the locking tabs 830 are engaged within the locking features 490, thus preventing any further sliding movement of the plunger depressor 700.

With the vein grafting preparation pump 300 in the locked, unused position, an irrigation syringe (not shown) may then be placed within the preparation pump 300. Specifically, the flange found at the base of typical syringe bodies is placed within the slot 450 at the front end 410 of the syringe holder 400. As such, the body of the syringe is thus held within the opening 440, with the majority of the syringe body and tip extending forwardly from the front end 410 of the syringe holder 400. Also, before the syringe is placed in the syringe holder 400, the syringe is filled with a fluid to be used in the irrigation of the vein harvested to be used in the grafting procedure. When filled, the plunger of the syringe is thereby drawn back a corresponding amount from within the syringe body. Thus, the distal end of the syringe plunger is engaged with the plunger depressor 700. The preparation pump 300 is then ready to be used to depress the syringe plunger so that the fluid within the syringe body is expressed from its tip and into the harvested vein.

Turning finally to FIG. 10, illustrated is the vein graft preparation pump 100 illustrated in FIG. 1 in a deployed, used state. As seen in this side view, in the deployed or used state, the plunger depressor 700 is shown on the right side of the pump 300, with the spring 600 released and stretching along the syringe holder 400.

More specifically, to deploy the plunger depressor 700 with a loaded syringe during use of the pump 300, the user operates the lock 800 to move it from the locked position to the unlocked position. In this illustrated embodiment, the lock 800 is moved to the unlocked position by rotating it forward, towards the front end 410. As discussed above, doing so disengages the locking tabs 830 from the features 490, thereby permitting slidable movement of the syringe depressor 700. In accordance with the disclosed principles, the previously compressed spring 600 releases its tension in constant and continuous manner. A vein grafting preparation pump 300 constructed in accordance with the disclosed principles incorporates a spring with a set spring constant (K) that is sufficient to deliver fluids from a syringe through a vessel cannula inserted into the vein section selected for the grafting procedure. This combination of regulated pressurization and continuity in the pressure applied through harvested veins results in a constant, safe pressure applied from the vein graft preparation pump to the harvested veins.

Additionally, the disclosed apparatus provides only a limited amount of pressure such that no injury is caused to the vein. Pressure is limited and maintained constant via use of the spring 600, which provides uniform pressurization at a predetermined amount safe for the harvested vein section, thereby eliminating the human error present with manual pressurization during distension of grafted veins. To do so, the spring constant (K) of the spring 600 is selected to provide the desired or required pressure, with a stiffer spring providing a greater amount of pressure, while a less stiff spring will provide a lower pressure of the fluid injected into the harvested vein for irrigation. The viscosity of the fluid used in preparing a vein graft can be considered in determining the desired amount of pressure provided by the spring 600. In some embodiments, an adjustment mechanism (not illustrated) may be provided in the pump 300, such as in the spring housing 500, that can adjust the distance between the backend of the spring 600 and the spring seat 550. Doing so would increase the pressure provided by the spring 600 during use of the pump 300, if needed or desired. Similarly, such a pressure adjustment mechanism could be used to decrease the pressure provided by the spring 600, by deceasing the distance between the rear end of the spring 600 and the spring seat 550. Such an adjustment mechanism may be useful when harvested veins of different wall thicknesses are being prepared, or if the viscosity of the irrigation fluid being used differs from one fluid to another, or to compensate for both of these variables.

Also, as described above, pump 300 as disclosed herein includes a locking mechanism (e.g., the lock 800) that allows delivery of fluids into the harvested vein to be stopped and started at any time. This locking/stopping capability also retracts the syringe plunger sufficiently to reduce or eliminate internal pressure to the vein if perforations in the vein are detected during irrigation/preparation. As such, any such perforations may be stitched or otherwise repaired while the pressure from the pump/syringe is eliminated by the locking/stopping mechanism. In the illustrated embodiment of FIGS. 9 and 10, the lock 800 is moved into the locking position by pivoting/rotating the lock 800 forward, towards the front end 410 of the pump 300. As the handle 820 is moved forward, the locking tabs 830 move rearwardly based on their location on the opposite side of the pivot axles in relation to the handle portion 820. As the locking tabs 830 are rotated rearwardly they will begin to engage the recesses 490 in the rails 420. When the lock 800 is about halfway through its possible pivot/rotation, the handle 820 is substantially perpendicular to the rails 420. This position alone ceases the sliding movement of the plunger depressor 700 when desired due to the full engagement of the locking tabs 830 with the recesses 490. However, advantageously, as the handle 820 is moved further forward, the locking tabs 830 more further reward, thus pushing against the recesses 490 such that the plunger depressor 700 is actually moved/slid back rearward slightly. This slight rearward movement of the plunger depressor 700 actually pulls the syringe plunger backwards slightly, which removes any pressure being applied from the plunger to the fluid within the syringe body.

Still further, in some embodiments of a preparation pump 300 in accordance with the disclosed principles, a fail-safe pressure release valve (not illustrated) may be included. Such a pressure release valve may be used to ensure a maximum pressure (which may be determined by the type/size of harvested vein, the type/viscosity of the fluid be used, or any other factor or combination of factors) is not exceeded when employing the pump 300 to prepare the harvested vein. Such a pressure release valve may take the form of valve formed on the syringe body, which may be turned to release pressure if needed. Additionally or alternatively, a damper (not illustrated) may also be positioned within the pump 300. An exemplary damper may employ a piston and accompanying fluid selected so that only a maximum amount of pressure can be applied with the movement of the syringe depressor 700 during use of the pump 300. A damper may also assist in ensuring that a consistent and uniform amount of pressure is provided by the pump 300, where a spring 600 alone may not provide the same amount of pressure at the beginning of its decompression than is applied at the end of its decompression. Also, such a damper could include the adjustment mechanism discussed above, which would permit a user of a pump 300 as disclosed herein to adjust the amount of pressure being applied during use, while also ensuring that amount of pressure does not exceed a maximum and is provided uniformly during use. Moreover, such a damper may be provided between the syringe depressor 700 and the front end 410 of the pump 300, or may be provided between the syringe depressor 700 and the spring housing 500. Of course, such a damper may be positioned in any advantageous location within the pump 300 without departing from the scope of the principles disclosed herein. Moreover, any other type of pressure regulating device, apparatus, or mechanism may be employed with a pump 300 as disclosed herein to set a safe, maximum pressure for the pump 300.

While this disclosure has been particularly shown and described with reference to preferred embodiments, it will be understood by those skilled in the pertinent field art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

The uses of the terms “a” and “an” and “the” and similar references in the context of describing the invention(s) (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. A vein graft preparation pump, comprising:
a syringe holder comprising:
a front end and a back end, the front end having a forward-facing opening configured to hold a syringe body and a slot formed within the forward-facing opening configured to receive a flange at a base of the syringe body,
a body comprising substantially parallel left side and right side rails connected to the front end and extending rearward from the front end, and
a support proximate the back end connecting rear ends of left side and right side rails;
a spring having a front end and a rear end;
a spring housing coupled to the rear ends of the left side and right side rails, and configured to secure the rear end of the spring therein; and
a plunger depressor having a back in contact with the front end of the spring, and a front having a plunger base configured to engage a distal end of a syringe plunger, the plunger depressor configured to laterally move from the front end to the back end of the syringe holder;
wherein when the plunger depressor is moved proximate the rear end of the syringe holder, the pump is in a loaded state where the spring is compressed between the plunger depressor and the spring housing, and wherein during use of the pump, decompression of the compressed spring causes movement of the plunger depressor towards the front end of the syringe holder thereby providing a substantially constant and uniform pressure depressing said syringe plunger.

2. The pump according to claim 1, further comprising a lock configured to stop the lateral movement of the plunger depressor.

3. The pump according to claim 2, wherein the lock is configured to pivot on the plunger depressor, the lock comprising one or more locking tabs formed on corresponding arms and sized to engage one or more corresponding locking features formed on the left side and right side rails.

4. The pump according to claim 3, wherein the plunger depressor further comprises a pair of pivot axles extending in opposing directions along a single axis of rotation for carrying the lock, wherein the lock comprises a pair of corresponding pivot holes formed on corresponding arms and configured to receive the corresponding pivot axles, and wherein the pivot axles and pivot holes cooperate to provide rotational movement of the lock about the axis of rotation.

5. The pump according to claim 4, wherein each of the pivot axles includes a corresponding axle stops formed on an exterior surface of each pivot axle, and wherein each of the pivot holes includes a corresponding pivot stop formed on an interior surface of each pivot hole, wherein corresponding axle stops and pivot stops are configured to contact each other to provide a positive stop for the rotational movement of the lock.

6. The pump according to claim 5, wherein when corresponding axle stops and pivot stops contact each other to provide a positive stop for the rotational movement of the lock, the rotational movement of the lock is stopped in a position where a locking tab is engaged within a corresponding locking feature so as to cause rearward movement of the plunger depressor.

7. The pump according to claim 1, wherein the syringe holder further comprises locking slots formed on the rear end of the syringe holder, and wherein the spring housing further comprises two or more extending locking arms, and a locking tab formed on an opposing, interior end of a corresponding locking arm, the locking tabs configured to be received in corresponding locking slots so as to secure the spring housing onto the rear end of the syringe holder.

8. The pump according to claim 1, wherein the spring housing further comprises lateral supports extending through a spring seat and extending outwardly to exterior walls of the spring housing, wherein portions of the lateral supports are sized to be received within alignment slots formed on rear ends of the left side and right side rails so as to prevent rotation of the syringe holder during use.

9. The pump according to claim 1, wherein the spring housing further comprises a spring seat configured to receive an inner diameter of the spring around it, and spring locks extending from the spring seat and configured to engage one or more coils of the spring to hold the spring within the spring housing.

10. The pump according to claim 1, wherein the plunger depressor further comprises a pair of opposing sidewalls configured to secure a distal end of a syringe plunger therebetween, and a raised feature sized and shaped to be received in a press-fit within the distal end of a plunger.

11. The pump according to claim 1, wherein the plunger depressor further comprises guiderails extending outwardly from opposing sides of the plunger depressor, wherein each guiderail is configured to be received in a corresponding alignment slot formed along each of the side rails.

12. A vein graft preparation pump, comprising:
a syringe holder comprising:
a front end and a back end, the front end having a forward-facing opening configured to hold a syringe body and a slot formed within the forward-facing opening configured to receive a flange at a base of the syringe body,
a body comprising substantially parallel left side and right side rails connected to the front end and extending rearward from the front end, and
a support proximate the back end connecting rear ends of left side and right side rails;
a spring having a front end and a rear end;
a spring housing coupled to the rear ends of the left side and right side rails, and configured to secure the rear end of the spring therein;
a plunger depressor having a back in contact with the front end of the spring, and a front having a plunger base configured to engage a distal end of a syringe plunger, the plunger depressor configured to laterally move from the front end to the back end of the syringe holder;
a lock configured to stop the lateral movement of the plunger depressor, the lock comprising one or more arms, each having a corresponding one or more locking tab sized to engage one or more corresponding locking features formed on the left side and right side rails;
wherein when the plunger depressor is moved proximate the rear end of the syringe holder, the pump is in a loaded state where the spring is compressed between the plunger depressor and the spring housing, and wherein during use of the pump, decompression of the compressed spring causes movement of the plunger depressor towards the front end of the syringe holder thereby providing a substantially constant and uniform pressure depressing said syringe plunger.

13. The pump according to claim 12, wherein the plunger depressor further comprises a pair of pivot axles extending in opposing directions along a single axis of rotation for carrying the lock, wherein the lock comprises a pair of corresponding pivot holes formed on corresponding arms and configured to receive the corresponding pivot axles, and wherein the pivot axles and pivot holes cooperate to provide rotational movement of the lock about the axis of rotation.

14. The pump according to claim 13, wherein when corresponding axle stops and pivot stops contact each other to provide a positive stop for the rotational movement of the lock, the rotational movement of the lock is stopped in a position where a locking tab is engaged within a corresponding locking feature so as to cause rearward movement of the plunger depressor.

15. The pump according to claim 12, wherein the syringe holder further comprises locking slots formed on the rear end of the syringe holder, and wherein the spring housing further comprises two or more extending locking arms, and a locking tab formed on an opposing, interior end of a corresponding locking arm, the locking tabs configured to be received in corresponding locking slots so as to secure the spring housing onto the rear end of the syringe holder.

16. The pump according to claim 12, wherein the spring housing further comprises lateral supports extending through a spring seat and extending outwardly to exterior walls of the spring housing, wherein portions of the lateral supports are sized to be received within alignment slots formed on rear ends of the left side and right side rails so as to prevent rotation of the syringe holder during use.

17. The pump according to claim 12, wherein the spring housing further comprises a spring seat configured to receive an inner diameter of the spring around it, and spring locks extending from the spring seat and configured to engage one or more coils of the spring to hold the spring within the spring housing.

18. The pump according to claim 12, wherein the plunger depressor further comprises a pair of opposing sidewalls configured to secure a distal end of a syringe plunger therebetween, and a raised feature sized and shaped to be received in a press-fit within the distal end of a plunger.

19. The pump according to claim 12, wherein the plunger depressor further comprises guiderails extending outwardly from opposing sides of the plunger depressor, wherein each guiderail is configured to be received in a corresponding alignment slot formed along each of the side rails.

20. The pump according to claim 12, further comprising one or more dampers configured to regulate the movement of the plunger depressor towards the front end of the syringe holder.
